(19) United States
(12) Patent Application Publication
(10) Pub. No.: US 2018/0021323 A1
(43) Pub. Date: Jan. 25, 2018

(54) FLIP - A SELECTIVE MOLECULAR TARGET OF SENESCENT CELLS

(71) Applicant: BIOVENTURES, LLC, Little Rock, AR (US)

(72) Inventors: Daohong Zhou, Little Rock, AR (US); Lijian Shao, Little Rock, AR (US); Wei Feng, Little Rock, AR (US); Jianhui Chang, Little Rock, AR (US); Yingying Wang, Little Rock, AR (US)

(21) Appl. No.: 15/545,480

(22) PCT Filed: Jan. 22, 2016
(86) PCT No.: PCT/US16/14518

§ 371 Date: Jul. 21, 2017

Related U.S. Application Data
(60) Provisional application No. 62/106,570, filed on Jan. 22, 2015.
(60) Provisional application No. 62/142,294, filed on Apr. 2, 2015.
(60) Provisional application No. 62/238,970, filed on Oct. 8, 2015.

(51) Int. Cl. A61K 31/45; A61K 31/4412; A61K 31/165; C12N 15/113; G01N 33/50

(52) U.S. Cl. CPC A61K 31/45; C12N 15/1137; G01N 33/5014; A61K 31/165; A61K 31/4412; C12N 2310/14; C12N 2310/531

(57) ABSTRACT

The present invention relates to modulators of FLIP protein and their method of use in the treatment and prevention of diseases and pathologies related to accumulation of senescent cells during aging, such as cancer, chronic obstructive pulmonary disease (COPD), osteoarthritis, atherosclerosis, neurodegenerative diseases, diabetes, and many others. The present invention also relates to pharmaceutical compositions containing these compounds as well as various uses thereof.

### CROSS REFERENCE TO RELATED APPLICATIONS

This application claims the benefit of U.S. Provisional Application No. 62/106,570, filed Jan. 22, 2015, U.S. Provisional Application No. 62/142,294, filed Apr. 2, 2015, and U.S. Provisional Application No. 62/238,970, filed Oct. 8, 2015, each of the disclosures of which are hereby incorporated by reference in their entirety.

### GOVERNMENTAL RIGHTS

This invention was made with government support under R01 CA122023 and R01 AI080421 awarded by the NIH. The government has certain rights in the invention.

### FIELD OF THE INVENTION

The present invention relates to modulators of FLIP protein and their method of use in the treatment and prevention of diseases and pathologies related to accumulation of senescent cells during aging, such as cancer, chronic obstructive pulmonary disease (COPD), osteoarthritis, atherosclerosis, neurodegenerative diseases, diabetes, and many others. The present invention also relates to pharmaceutical compositions containing these compounds as well as various uses thereof.

### BACKGROUND OF THE INVENTION

Age is a leading risk factor for many human diseases, including most cancers, atherosclerosis, neurodegenerative diseases, diabetes, and many others. Additionally, numerous diseases are caused by accelerated aging and/or defects in DNA damage report and telomere maintenance such as Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, and others. An increasing body of evidence demonstrates that aging is associated with an accumulation of senescent cells. When a cell becomes senescent, it loses its reproductive function, which may cause tissue degeneration. In addition, senescent cells produce increased levels of free radical and various inflammatory mediators that can induce tissue damage and cell transformation. Therefore, selective depletion of senescent cells may be a novel anti-aging strategy that may prevent cancer and various human diseases associated with aging and rejuvenate the body to live a healthier lifespan. This assumption is supported by a recent study showing that selective depletion of senescent cells in the BubR1 progeroid mouse model by a genetic approach resulted in the delay of various age-related pathologies and disorders. However, there is no drug that can selectively deplete senescent cells. Therefore, a method to selectively deplete senescent cells is needed.

### SUMMARY OF THE INVENTION

In an aspect, the present disclosure encompasses a method of selectively killing one or more senescent cells in a subject in need thereof. The method comprises administering to the subject a composition comprising a compound that modulates c-Fas-associated death domain-like interleukin-1 converting enzyme-like inhibitory protein (FLIP).

In another aspect, the present disclosure encompasses a method for delaying at least one feature of aging in a subject. The method comprises administering a composition comprising a therapeutically effective amount of a compound that modulates FLIP.

In still another aspect, the present disclosure encompasses a method of treating an age-related disease or condition. The method comprises administering a composition comprising a therapeutically effective amount of compound that modulates FLIP, provided the age-related disease or condition is not cancer.

In still yet another aspect, the present disclosure encompasses a method of treating a senescence-associated disease or condition. The method comprises administering a composition comprising a therapeutically effective amount of compound that modulates FLIP, provided the senescence-associated disease or condition is not cancer.

### BRIEF DESCRIPTION OF THE FIGURES

The application file contains at least one photograph executed in color. Copies of this patent application publication with color photographs will be provided by the Office upon request and payment of the necessary fee.

FIG. 1A, FIG. 1B and FIG. 1C depict immunoblots showing that IR-induced and replicative senescent cells express increased levels of FLIP and Fas. (FIG. 1A) Expression of FLIP, XIAP, cIAP1, cIAP2 and β-actin was analyzed by Western blots in control (CTL) and WI38 human fibroblast cells 1, 3, 5, 7 and 10 days after exposure to 10 Gy γ-irradiation. (FIG. 1B) Expression of Fas, DR5, TNF-R1 and β-actin was analyzed by Western blots in control (CTL) and WI38 human fibroblast cells 1, 3, 5, 7 and 10 days after exposure to 10 Gy γ-irradiation. (FIG. 1C) Expression of FLIP, XIAP, cIAP1, cIAP2, Fas, DR5, FADD, TNF-R1 and β-actin was analyzed by Western blots in control (CTL) and replicative senescent WI38 human fibroblast cells. The results showed that both IR-induced and replicative senescent cells (SC) expressed increased levels of FLIP and Fas as compared with control cells.

FIG. 2A, FIG. 2B, FIG. 2C and FIG. 2D depicts images, immunoblots and graphs showing knockdown of FLIP expression selectively kills senescent cells. (FIG. 2A) Treatment with doxycycoline (DOX) dose-dependently induces FLIP-shRNA expression in WI38 cell line after the cells were stably transfected with a plasmid containing FLIP-shRNA and red fluorescent protein (RFP) genes. (FIG. 2B) Induction of FLIP-shRNA by doxycycoline down-regulates FLIP expression in IR-induced senescent WI38 cells stably transfected with FLIP-shRNA but not in vector transfected cells. (FIG. 2C, FIG. 2D) Induction of FLIP-shRNA expression by DOX selectively kills IR-induced senescent cells (SC; FIG. 2D) in a DOX-dose-dependent manner but has minimal effect on normal cells (NC; FIG. 2C).

FIG. 3A, FIG. 3B, FIG. 3C, FIG. 3D and FIG. 3E depicts immunoblots and graphs showing that down-regulation of FLIP expression by a small molecule selectively kill senescent cells. (FIG. 3A) IR-induced senescent (SC) WI38 cells exhibit an increased expression of FLIP, which was down-regulated after treatment with droxinostat (Drox). The expression of FLIP in normal (NC) and IR-induced senescent (SC) WI38 cells was determined by Western blot 24 h after they were treated with vehicle (VEH) or Drox (10 μM). (FIG. 3B) Treatment with droxinostat (Drox) down-regulates the expression of FLIP mRNA in IR-induced senescent (SC) WI38 cells. IR-induced senescent (SC) WI38 cells were treated with vehicle (VEH) or Drox (10 μM) for 6 h and the expression of FLIP mRNA in these cells was determined by qPCR. (FIG. 3C, FIG. 3D) Droxinostat (Drox) selectively kills IR-induced senescent (SC; FIG. 3D) WI38 cells but has minimal effect on normal (NC; FIG. 3C) WI38 cells. Viable cells were determined 72 h after normal (NC) and IR-induced senescent (SC) WI38 cells were treated with vehicle (VEH) or increasing concentrations of Drox. The data are presented as a percentage of control cells treated with VEH. (FIG. 3E) Treatment with droxinostat (Drox), piperlongumine (PL) and EF-24 down-regulates the expression of FLIP in IR-induced senescent (SC) WI38 cells. Normal (NC) and IR-induced senescent (SC) WI38 cells were treated with vehicle (VEH), Drox (10 μM), PL (10 μM), and EF-24 (10 μM) for 24 h and the expression of FLIP in these cells was determined by Western blots.

### DETAILED DESCRIPTION OF THE INVENTION

Applicants have discovered that c-Fas-associated death domain-like interleukin-1 converting enzyme-like inhibitory protein (FLIP) is upregulated when a cell undergoes senescence. It functions as an anti-apoptotic protein to inhibit the activation of caspase 8 to prevent apoptosis. The applicants have discovered senolytic drugs that down-regulate FLIP in senescent cells. The down-regulation is associated with induction of senescent cell apoptosis. Accordingly the present disclosure provides compositions and methods for selectively depleting senescent cells. Additional aspects of the invention are described below.

### I. Compositions

In an aspect, a composition of the invention comprises a compound that modulates FLIP. Specifically, a compound that modulates FLIP may be a compound that downregulates FLIP. FLIP may also be referred to as c-FLIP, Casper, iFLICE, FLAME-1, CASJ, CLARP, MRIT or usurpin. A compound of the invention may be modified to improve potency, bioavailability, solubility, stability, handling properties, or a combination thereof, as compared to an unmodified version.

A composition of the invention may optionally comprise one or more additional drug or therapeutically active agent in addition to a compound that modulates FLIP. For example, a composition of the invention may optionally comprise one or more compounds that interact with FAS and/or DRs and induce apoptosis and/or Bcl-2 inhibitors. Specifically, a composition of the invention may optionally comprise one or more compounds that interact with FAS and/or DRs and induce apoptosis as described in U.S. 62/106,573 and/or Bcl-2 inhibitors as described in PCT/US2015/029208, the disclosures of which are hereby incorporated by reference in their entirety. Still further, a composition of the invention may optionally comprise one or more piperlongumines or derivatives thereof. Specifically, a composition of the invention may optionally comprise one or more piperlongumines or derivatives thereof as described in PCT/US2015/041470, the disclosure of which is hereby incorporated by reference in its entirety. A composition of the invention may further comprise a pharmaceutically acceptable excipient, carrier or diluent. Further, a composition of the invention may contain preserving agents, solubilizing agents, stabilizing agents, wetting agents, emulsifiers, sweeteners, colorants, odorants, salts (substances of the present invention may themselves be provided in the form of a pharmaceutically acceptable salt), buffers, coating agents or antioxidants.

Other aspects of the invention are described in further detail below.

(a) Compound that Modulates FLIP

In general, the compounds detailed herein include compounds that modulate FLIP. Specifically, a compound that modulates FLIP may be a compound that downregulates FLIP. Methods to determine if a compound modulates FLIP are known in the art. For example, FLIP nucleic acid expression, FLIP protein expression, or FLIP activity may be measured as described in more detail below.

A compound with the ability to modulate FLIP in senescent cells may potentially be used as a senolytic drug. A senolytic drug may include, without limitation, a compound, a drug, a small molecule, a peptide, a nucleic acid molecule, a protein, an antibody, and combinations thereof. A nucleic acid molecule may be an antisense oligonucleotide, a ribozyme, a small nuclear RNA (snRNA), a long noncoding RNA (LncRNA), or a nucleic acid molecule which forms triple helical structures. Non-limiting examples of a compound that modulates FLIP include histone deacetylase inhibitors, such as droxinostat (4-(4-chloro-2-methylphenoxy)-N-hydroxybutanamide), valproic acid, trichostatin, SAHA, vorinostat, or a derivative thereof; piperlongumine or a derivative thereof; curcumin or a derivative thereof such as EF-24, 1-(3,5-di-tert-butyl-4-hydroxyphenyl)-2-(3-ethyl-2-imino-2,3-dihydro-1H-benzimidazol-1-yl)ethanone hydrochloride; anisomycin, taurolidine, obtusaquinone, bortezomib (PS-34), nutlin-3, honokol, berberine, genistein, celecoxib, cisplatin, oxaliplatin, doxorubicin, camptothecin, 9-NC, irinotecan, Lupeol (triterpene), celastrol, zerumbone (sesquiterpene), withaferin A (steroidal lactone), quinacrine, chrysin (flavanoid), CDDO-imadazolide (synthetic triterpenoid), siRNAs, actinomycin D, cyclohexamide, fluorouracil (5-FU), MG-132, troglitazone, sorafenib, Taxol (paclitaxel), nocodazole, genistein (isoflavone), silibinin (flavonoid), OH14 and CDDO-Me. For additional compounds, see for example WO2011/130395, Schimmer A D et al. Cancer Res. 2006; 66:2367-75, Mawji I A et al. Cancer Res. 2007; 67:8307-15, Shirley S & Micheau O. Cancer Letter 2013:332:141-50, Sanders Y Y et al. Redox Biol. 2013; 1:8-16, Safa A R & Pollok K E. Cancer 2011; 3:1639-71, Raja S M et al. Mol Cancer Ther. 2008; 7:2212-23, Lee S-J et al. Int J Oncol. 2011; 38:485-492, Siegelin M D et al. Neuroscie Lett. 2009; 453:92-7, Chen S et al. Cancer Res. 2011; 71:6270-81, US 20050084876, US 20050208151, each of which is hereby incorporated by reference in its entirety. In certain embodiments, a compound that modulates FLIP may be an HDAC inhibitor. In a specific embodiment, a compound that modulates FLIP may be droxinostat or a derivative thereof. In another specific embodiment, a compound that modulates FLIP may be piperlongumine or a derivative thereof. In still another specific embodiment, a compound that modulates FLIP may be curcumin or a derivative thereof. In still yet another specific embodiment, a compound that modulates FLIP may be EF-24 or a derivative thereof. In a different embodiment, a compound that modulates FLIP may be OH14 or a derivative thereof.

Dosages of a compound that modulates FLIP can vary between wide limits, depending upon the disease or disorder to be treated and/or the age and condition of the subject to be treated. In an embodiment where a composition comprising a compound that modulates FLIP is contacted with a sample, the concentration of the compound that modulates FLIP may be from about 1 μM to about 40 μM. Alternatively, the concentration of the compound that modulates FLIP may be from about 5 μM to about 25 μM. For example, the concentration of the compound that modulates FLIP may be about 1, about 2.5 about 5, about 6, about 7, about 8, about 9, about 10, about 11, about 12, about 12, about 14, about 15, about 16, about 17, about 18, about 19, about 20, about 21, about 22, about 23, about 24, about 25, about 30, about 35, or about 40 μM. Additionally, the concentration of the compound that modulates FLIP may be greater than 40 μM. For example, the concentration of the compound that modulates FLIP may be about 40, about 45, about 50, about 55, about 60, about 65, about 70, about 75, about 80, about 85, about 90, about 95 or about 100 μM. In certain embodiments, the concentration of the compound that modulates FLIP may be from about 1 μM to about 10 μM, from about 10 μM to about 20 μM, from about 20 μM to about 30 μM, or from about 30 μM to about 40 μM. In a specific embodiment, the concentration of the compound that modulates FLIP may be from about 1 μM to about 10 μM.

In an embodiment where the composition comprising a compound that modulates FLIP is administered to a subject, the dose of the compound that modulates FLIP may be from about 0.1 mg/kg to about 500 mg/kg. For example, the dose of the compound that modulates FLIP may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of the compound that modulates FLIP may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of the compound that modulates FLIP may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

i. FLIP Nucleic Acid Expression

In an embodiment, FLIP nucleic acid expression may be measured to identify a compound that modulates FLIP. For example, when FLIP nucleic acid expression is decreased in the presence of a compound relative to an untreated control, the compound downregulates FLIP. In a specific embodiment, FLIP mRNA may be measured to identify a compound that modulates FLIP.

Methods for assessing an amount of nucleic acid expression in cells are well known in the art, and all suitable methods for assessing an amount of nucleic acid expression known to one of skill in the art are contemplated within the scope of the invention. The term “amount of nucleic acid expression” or “level of nucleic acid expression” as used herein refers to a measurable level of expression of the nucleic acids, such as, without limitation, the level of messenger RNA (mRNA) transcript expressed or a specific variant or other portion of the mRNA, the enzymatic or other activities of the nucleic acids, and the level of a specific metabolite. The term “nucleic acid” includes DNA and RNA and can be either double stranded or single stranded. Non-limiting examples of suitable methods to assess an amount of nucleic acid expression may include arrays, such as microarrays, PCR, such as RT-PCR (including quantitative RT-PCR), nuclease protection assays and Northern blot analyses. In a specific embodiment, determining the amount of expression of a target nucleic acid comprises, in part, measuring the level of target nucleic acid mRNA expression.

In one embodiment, the amount of nucleic acid expression may be determined by using an array, such as a microarray. Methods of using a nucleic acid microarray are well and widely known in the art. For example, a nucleic acid probe that is complementary or hybridizable to an expression product of a target gene may be used in the array. The term “hybridize” or “hybridizable” refers to the sequence specific non-covalent binding interaction with a complementary nucleic acid. In a preferred embodiment, the hybridization is under high stringency conditions. Appropriate stringency conditions which promote hybridization are known to those skilled in the art, or can be found in Current Protocols in Molecular Biology, John Wiley & Sons, N.Y. (1989), 6.3.1 6.3.6. The term “probe” as used herein refers to a nucleic acid sequence that will hybridize to a nucleic acid target sequence. In one example, the probe hybridizes to an RNA product of the nucleic acid or a nucleic acid sequence complementary thereof. The length of probe depends on the hybridization conditions and the sequences of the probe and nucleic acid target sequence. In one embodiment, the probe is at least 8, 10, 15, 20, 25, 50, 75, 100, 150, 200, 250, 400, 500 or more nucleotides in length.

In another embodiment, the amount of nucleic acid expression may be determined using PCR. Methods of PCR are well and widely known in the art, and may include quantitative PCR, semi-quantitative PCR, multiplex PCR, or any combination thereof. Specifically, the amount of nucleic acid expression may be determined using quantitative RT-PCR. Methods of performing quantitative RT-PCR are common in the art. In such an embodiment, the primers used for quantitative RT-PCR may comprise a forward and reverse primer for a target gene. The term “primer” as used herein refers to a nucleic acid sequence, whether occurring naturally as in a purified restriction digest or produced synthetically, which is capable of acting as a point of synthesis when placed under conditions in which synthesis of a primer extension product, which is complementary to a nucleic acid strand is induced (e.g. in the presence of nucleotides and an inducing agent such as DNA polymerase and at a suitable temperature and pH). The primer must be sufficiently long to prime the synthesis of the desired extension product in the presence of the inducing agent. The exact length of the primer will depend upon factors, including temperature, sequences of the primer and the methods used. A primer typically contains 15-25 or more nucleotides, although it can contain less or more. The factors involved in determining the appropriate length of primer are readily known to one of ordinary skill in the art.

The amount of nucleic acid expression may be measured by measuring an entire mRNA transcript for a nucleic acid sequence, or measuring a portion of the mRNA transcript for a nucleic acid sequence. For instance, if a nucleic acid array is utilized to measure the amount of mRNA expression, the array may comprise a probe for a portion of the mRNA of the nucleic acid sequence of interest, or the array may comprise a probe for the full mRNA of the nucleic acid sequence of interest. Similarly, in a PCR reaction, the primers may be designed to amplify the entire cDNA sequence of the nucleic acid sequence of interest, or a portion of the cDNA sequence. One of skill in the art will recognize that there is more than one set of primers that may be used to amplify either the entire cDNA or a portion of the cDNA for a nucleic acid sequence of interest. Methods of designing primers are known in the art. Methods of extracting RNA from a biological sample are known in the art.

The level of expression may or may not be normalized to the level of a control nucleic acid. Such a control nucleic acid should not specifically hybridize with an aiRNA nucleotide sequence of the invention. This allows comparisons between assays that are performed on different occasions.

ii. FLIP Protein Expression

In another embodiment, FLIP protein expression may be measured to identify a compound that modulates FLIP. For example, when FLIP protein expression is decreased in the presence of a compound relative to an untreated control, the compound downregulates FLIP. In a specific embodiment, FLIP protein expression may be measured using immunoblot.

Methods for assessing an amount of protein expression are well known in the art, and all suitable methods for assessing an amount of protein expression known to one of skill in the art are contemplated within the scope of the invention. Non-limiting examples of suitable methods to assess an amount of protein expression may include epitope binding agent-based methods and mass spectrometry based methods.

In some embodiments, the method to assess an amount of protein expression is mass spectrometry. By exploiting the intrinsic properties of mass and charge, mass spectrometry (MS) can resolve and confidently identify a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can use mass spectrometry to look for the level of protein encoded from a target nucleic acid of the invention.

In some embodiments, the method to assess an amount of protein expression is an epitope binding agent-based method. As used herein, the term “epitope binding agent” refers to an antibody, an aptamer, a nucleic acid, an oligonucleic acid, an amino acid, a peptide, a polypeptide, a protein, a lipid, a metabolite, a small molecule, or a fragment thereof that recognizes and is capable of binding to a target gene protein. Nucleic acids may include RNA, DNA, and naturally occurring or synthetically created derivative.

As used herein, the term “antibody” generally means a polypeptide or protein that recognizes and can bind to an epitope of an antigen. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art (See, e.g. U.S. Pat. No. 7,939,313; herein incorporated by reference in its entirety).

In general, an epitope binding agent-based method of assessing an amount of protein expression comprises contacting a sample comprising a polypeptide with an epitope binding agent specific for the polypeptide under conditions effective to allow for formation of a complex between the epitope binding agent and the polypeptide. Epitope binding agent-based methods may occur in solution, or the epitope binding agent or sample may be immobilized on a solid surface. Non-limiting examples of suitable surfaces include microtitre plates, test tubes, beads, resins, and other polymers.

An epitope binding agent may be attached to the substrate in a wide variety of ways, as will be appreciated by those in the art. The epitope binding agent may either be synthesized first, with subsequent attachment to the substrate, or may be directly synthesized on the substrate. The substrate and the epitope binding agent may be derivatized with chemical functional groups for subsequent attachment of the two. For example, the substrate may be derivatized with a chemical functional group including, but not limited to, amino groups, carboxyl groups, oxo groups or thiol groups. Using these functional groups, the epitope binding agent may be attached directly using the functional groups or indirectly using linkers.

The epitope binding agent may also be attached to the substrate non-covalently. For example, a biotinylated epitope binding agent may be prepared, which may bind to surfaces covalently coated with streptavidin, resulting in attachment. Alternatively, an epitope binding agent may be synthesized on the surface using techniques such as photopolymerization and photolithography. Additional methods of attaching epitope binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, both of which are hereby incorporated by reference in their entirety).

Contacting the sample with an epitope binding agent under effective conditions for a period of time sufficient to allow formation of a complex generally involves adding the epitope binding agent composition to the sample and incubating the mixture for a period of time long enough for the epitope binding agent to bind to any antigen present. After this time, the complex will be washed and the complex may be detected by any method well known in the art. Methods of detecting the epitope binding agent-polypeptide complex are generally based on the detection of a label or marker. The term “label”, as used herein, refers to any substance attached to an epitope binding agent, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni2+, Flag tags, myc tags, heavy metals, and enzymes (including alkaline phosphatase, peroxidase, and luciferase). Methods of detecting an epitope binding agent-polypeptide complex based on the detection of a label or marker are well known in the art.

In some embodiments, an epitope binding agent-based method is an immunoassay. Immunoassays can be run in a number of different formats. Generally speaking, immunoassays can be divided into two categories: competitive immunoassays and non-competitive immunoassays. In a competitive immunoassay, an unlabeled analyte in a sample competes with labeled analyte to bind an antibody. Unbound analyte is washed away and the bound analyte is measured. In a non-competitive immunoassay, the antibody is labeled, not the analyte. Non-competitive immunoassays may use one antibody (e.g. the capture antibody is labeled) or more than one antibody (e.g. at least one capture antibody which is unlabeled and at least one “capping” or detection antibody which is labeled.) Suitable labels are described above.

In some embodiments, the epitope binding agent-based method is an ELISA. In other embodiments, the epitope binding agent-based method is a radioimmunoassay. In still other embodiments, the epitope binding agent-based method is an immunoblot or Western blot. In alternative embodiments, the epitope binding agent-based method is an array. In another embodiment, the epitope binding agent-based method is flow cytometry. In different embodiments, the epitope binding agent-based method is immunohistochemistry (IHC). IHC uses an antibody to detect and quantify antigens in intact tissue samples. The tissue samples may be fresh-frozen and/or formalin-fixed, paraffin-embedded (or plastic-embedded) tissue blocks prepared for study by IHC. Methods of preparing tissue block for study by IHC, as well as methods of performing IHC are well known in the art.

iii. FLIP Activity

In an embodiment, FLIP activity may be measured to identify a compound that modulates FLIP. FLIP inhibits the activation of caspase 8 to prevent apoptosis. Accordingly, apoptosis may be measured as an indication of FLIP activity. Apoptosis may be measured using methods standard in the art as described below in Section II(c). For example, when apoptosis of senescent cells is increased in the presence of a compound relative to an untreated control, the compound downregulates FLIP.

In another embodiment, cell viability may be measured as an indication of FLIP activity. Cell viability may be measured using methods standard in the art as described below in Section II(c). For example, when cell viability of senescent cells is decreased in the presence of a compound relative to an untreated control, the compound downregulates FLIP.

In still another embodiment, caspases may be measured as an indication of FLIP activity. Specifically, caspase 8 may be measured as an indication of FLIP activity. In the absence of FLIP, procaspase-8 dimerization induces full processing and activation of caspase-8, leading to the release of active caspase-8 to the cytosol and activation of apoptosis. In the presence of FLIP, procaspase-8 remains mostly uncleaved and thus non-functional. Caspases may be measured using, for example, methods to detect protein expression as described above. For example, when caspase-8 expression in senescence cells is increased in the presence of a compound relative to an untreated control, the compound downregulates FLIP.

In still yet another embodiment, downstream effectors of FLIP may be measured. For example, FLIP inhibits procaspase-8 dimerization and activation, thus blocking the activation of the apoptotic cascade. Accordingly, other proteins involved in the apoptotic cascade may be measured as an indication of FLIP activity. Non-limiting examples include caspase-10, caspase-4, caspase-9, caspase-7, caspase-3, caspase-6, RIP1, Bid, Bcl-XL, Bcl-2, Bak, Bax, IAP, XIAP, CytC and SMAC. The above list included both cleaved proteins (i.e. p43/41 caspase-8) and “pro” proteins (i.e. procaspase-3). Additionally, c-Fos and NF-κB are known to be affected by FLIP. Thus, c-Fos and NF-κB may be measured as an indication of FLIP activity.

### (b) Components of the Composition

The present disclosure also provides pharmaceutical compositions. The pharmaceutical composition comprises a compound that modulates FLIP, as an active ingredient, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, and combinations thereof.

In another embodiment, the excipient may be a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In still another embodiment, the excipient may be a buffering agent. Representative examples of suitable buffering agents include, but are not limited to, phosphates, carbonates, citrates, tris buffers, and buffered saline salts (e.g., Tris buffered saline or phosphate buffered saline).

In various embodiments, the excipient may be a pH modifier. By way of non-limiting example, the pH modifying agent may be sodium carbonate, sodium bicarbonate, sodium citrate, citric acid, or phosphoric acid.

In a further embodiment, the excipient may be a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In another alternate embodiment, the excipient may be a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; and antimicrobials, such as parabens, chlorobutanol, or phenol.

In a further embodiment, the excipient may be a lubricant. Non-limiting examples of suitable lubricants include minerals such as talc or silica; and fats such as vegetable stearin, magnesium stearate or stearic acid.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Flavoring agents may be chosen from synthetic flavor oils and flavoring aromatics and/or natural oils, extracts from plants, leaves, flowers, fruits, and combinations thereof.

In still a further embodiment, the excipient may be a coloring agent. Suitable color additives include, but are not limited to, food, drug and cosmetic colors (FD&C), drug and cosmetic colors (D&C), or external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

The composition can be formulated into various dosage forms and administered by a number of different means that will deliver a therapeutically effective amount of the active ingredient. Such compositions can be administered orally (e.g. inhalation), parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, intra-articular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980). In a specific embodiment, a composition may be a food supplement or a composition may be a cosmetic.

Solid dosage forms for oral administration include capsules, tablets, caplets, pills, powders, pellets, and granules. In such solid dosage forms, the active ingredient is ordinarily combined with one or more pharmaceutically acceptable excipients, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof. For administration by inhalation, the compounds are delivered in the form of an aerosol spray from pressured container or dispenser which contains a suitable propellant, e.g., a gas such as carbon dioxide, or a nebulizer.

For parenteral administration (including subcutaneous, intradermal, intravenous, intramuscular, intra-articular and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising a compound that modulates FLIP is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of a compound that modulates FLIP in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, a compound that modulates FLIP may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying a compound that modulates FLIP (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. A compound that modulates FLIP may be encapsulated in a microemulsion by any method generally known in the art.

In yet another embodiment, a compound that modulates FLIP may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### (c) Bcl-2 Family Inhibitor

In an aspect, the composition further comprises at least one inhibitor of one or more anti-apoptotic proteins in the Bcl-2 family. Members of the B-cell lymphoma 2 (Bcl-2) family control the integrity of the outer mitochondrial membrane (OMM) and thus are critical in determining the susceptibility of cells to apoptosis induced by the intrinsic pathway. Bcl-2 family members can be divided into three subfamilies based on structural and functional features: an anti-apoptotic family, a multidomain pro-apoptotic family, and a BH3-only pro-apoptotic family. The anti-apoptotic subfamily suppresses apoptosis and promotes cell survival but not cell proliferation. As such, the anti-apoptotic proteins in the Bcl-2 family may also be referred to as pro-survival proteins. Non-limiting examples of anti-apoptotic Bcl-2 family proteins may include Bcl-2, Bcl-xL, Bcl-w, Mcl-1, Bfl1/A-1, and Bcl-B. The anti-apoptotic Bcl-2 family proteins are characterized by the presence of up to four relatively short sequence motifs, which are less than 20 amino acids in length, known as Bcl-2 homology 1 (BH1), BH2, BH3 and BH4 domains. They also have a C-terminal membrane-anchoring sequence and a similar three-dimensional structure. Inhibitors of one or more anti-apoptotic proteins in the Bcl-2 family may promote cell death by antagonizing the pro-survival function of the Bcl-2 protein family thereby inducing apoptosis. A Bcl-2 family inhibitor may inhibit one or more anti-apoptotic proteins in the Bcl-2 family. In an exemplary embodiment, a Bcl-2 family inhibitor is a Bcl-2, Bcl-xL and Bcl-w inhibitor.

An inhibitor of one or more anti-apoptotic proteins in the Bcl-2 family may be an inhibitor that inhibits nucleic acid expression, protein expression, or protein function of a Bcl-2 family protein. An inhibitor may selectively inhibit one, two, three, four, five, six or more members of the Bcl-2 family proteins. In an embodiment, an inhibitor may affect nucleic acid or protein expression of a Bcl-2 family protein. Non-limiting examples of inhibitors that decrease nucleic acid and protein expression may include histone deacetylase inhibitors such as sodium butyrate and depsipeptide, synthetic cytotoxic retinoid such as fenretinide, and cyclin-dependent kinase inhibitors such as flavopiridol. Alternatively, an inhibitor may be an antisense molecule. For example, oblimersen sodium (G3139) is a Bcl-2 antisense that targets BCL-2 mRNA. In another embodiment, an inhibitor may be a natural inhibitor of Bcl-2 family interactions. For example, progidiosin molecules (bypyrrole-containing natural products), such as GX15-070 (obatoclax) may inhibit Bcl-2 family proteins such as Bcl-2, Bcl-XL, Bcl-w and Mcl-1. Additionally, the natural inhibitor gossypol (AT-101) and its derivatives, apogossypolone, TW37, TM-1206, BM-1074 and BM-1197 may inhibit Bcl-2 family proteins such as Bcl-2, Bcl-XL, and Mcl-1. In still another embodiment, an inhibitor may be designed to bind the hydrophobic grove of anti-apoptotic Bcl-2 family proteins in place of BH3-only proteins (i.e., BH3-mimetics). As such, an inhibitor may be a small molecule inhibitor of one or more anti-apoptotic proteins in the Bcl-2 family. For example, isoxazolidine-based small molecules that interact with Bcl-2 and Bcl-XL, ABT-737 and ABT-263 that bind Bcl-2, Bcl-XL, and Bcl-w. Non-limiting examples of other Bcl-2 family inhibitors may include SAHB_{A}, terphenyl, benzoylureas, A-385358, HA-14, antimycin A, ABT199, WEHI539, MIM-1, and BH₃Is. In a preferred embodiment, an inhibitor is a molecule similar to ABT-263. In an exemplary embodiment, an inhibitor of one or more anti-apoptotic proteins in the Bcl-2 family is ABT-263 (navitoclax).

In an aspect, a composition of the invention further comprises ABT-263, an ABT-263 analog or an ABT-263 derivative. ABT-263, ABT-263 analogs or ABT-263 derivatives may be modified to improve bioavailability, solubility, stability, handling properties, or a combination thereof, as compared to an unmodified version. Thus, in another aspect, a composition of the invention may further comprise modified ABT-263, ABT-263 analog or ABT-263 derivative. In still another aspect, a composition of the invention further comprises a prodrug of ABT-263, an ABT-263 analog or an ABT-263 derivative.

In an embodiment, the composition further comprises at least one inhibitor of one or more anti-apoptotic proteins in the Bcl-2 family. For example, the composition may further comprise 1, 2, 3, 4 or 5 or more inhibitors of one or more anti-apoptotic proteins in the Bcl-2 family. Each Bcl-2 inhibitor of the composition may target the same or different anti-apoptotic protein in the Bcl-2 family. In an embodiment, the composition may further comprise two inhibitors of one or more anti-apoptotic proteins in the Bcl-2 family. In another embodiment, the composition may further comprise one inhibitor of one or more anti-apoptotic proteins in the Bcl-2 family.

Dosages of the Bcl-2 family inhibitor can vary between wide limits, depending upon the disease or disorder to be treated, the age and condition of the subject to be treated. In an embodiment where the composition further comprising at least one inhibitor of one or more anti-apoptotic proteins in the Bcl-2 family is contacted with a sample, the concentration of the at least one inhibitor of one or more anti-apoptotic proteins in the Bcl-2 family may be from about 0.01 μM to about 10 μM. Alternatively, the concentration of the at least one inhibitor of one or more anti-apoptotic proteins in the Bcl-2 family may be from about 0.01 μM to about 5 μM. For example, the concentration of the at least one inhibitor of one or more anti-apoptotic proteins in the Bcl-2 family may be about 0.01, about 0.05, about 0.1, about 0.2, about 0.3, about 0.4, about 0.5, about 0.6, about 0.7, about 0.8, about 0.9, about 1, about 2, about 3, about 4, about 5, about 6, about 7, about 8, about 9 or about 10 μM. Additionally, the concentration of the at least one inhibitor of one or more anti-apoptotic proteins in the Bcl-2 family may be greater than 10 μM. For example, the concentration of the at least one inhibitor of one or more anti-apoptotic proteins in the Bcl-2 family may be about 10, about 15, about 20, about 25, about 30, about 35, about 40, about 45, about 50, about 55, about 60, about 65, about 70, about 75, about 80, about 85, about 90, about 95 or about 100 μM.

In an embodiment where the composition further comprising at least one inhibitor of one or more anti-apoptotic proteins in the Bcl-2 family is administered to a subject, the dose of inhibitor may be from about 0.1 mg/kg to about 500 mg/kg. For example, the dose of the least one inhibitor of one or more anti-apoptotic proteins in the Bcl-2 family may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of the least one inhibitor of one or more anti-apoptotic proteins in the Bcl-2 family may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, the dose of the least one inhibitor of one or more anti-apoptotic proteins in the Bcl-2 family may be about 300 mg/kg, about 325 mg/kg, about 350 mg/kg, about 375 mg/kg, about 400 mg/kg, about 425 mg/kg, about 450 mg/kg, about 475 mg/kg or about 500 mg/kg.

### II. Methods

The present disclosure encompasses a method of selectively killing one or more senescent cells in a sample, the method comprising contacting a composition comprising an effective amount of a compound that modulates FLIP with the sample. In another aspect, the present disclosure encompasses a method of selectively killing one or more senescent cells in a subject in need thereof, the method comprising administering to the subject a composition comprising a therapeutically effective amount of a compound that modulates FLIP.

By selectively killing one or more senescent cells is meant a composition of the invention does not appreciably kill non-senescent cells at the same concentration. Accordingly, the median lethal dose or LD50 of the composition in non-senescent cells may be about 2 to about 50 times higher than the LD50 of the composition in senescent cells. As used herein, the LD50 is the concentration of composition required to kill half the cells in the cell sample. For example, the LD50 of the composition in non-senescent cells may be greater than about 2, about 3, about 4, about 5, about 6, about 7, about 8, about 9 or about 10 times higher than the LD50 of the composition in senescent cells. Alternatively, the LD50 of the composition in non-senescent cells may be greater than about 10, about 15, about 20, about 25, about 30, about 35, about 40, about 45, or about 50 times higher than the LD50 of the composition in senescent cells. Additionally, the LD50 of the composition in non-senescent cells may be greater than 50 times higher than the LD50 of the composition in senescent cells. In certain embodiments, the LD50 of the composition in non-senescent cells is about 2 to about 10 times higher than the LD50 of the composition in senescent cells. In an exemplary embodiment, the LD50 of the composition in non-senescent cells is about 3 to about 6 times higher than the LD50 of the composition in senescent cells.

The progression from an actively dividing cell to a metabolically active, non-dividing cell is termed “senescence” or “cellular senescence.” As used herein, the terms “senescence” and “cellular senescence” may be used interchangeably. The term “senescence” also refers to the state into which cells enter after multiple rounds of division and, as a result of cellular pathways, future cell division is prevented from occurring even though the cell remains metabolically active. Senescent cells may differ from their pre-senescent counterparts in one or more of the following ways: 1) they arrest growth and cannot be stimulated to reenter the cell cycle by physiological mitogens; 2) they become resistant to apoptotic cell death; and/or 3) they acquire altered differentiated functions.

In contrast to cancer cells which grow and divide uncontrollably, the ability of most differentiated eukaryotic cells to proliferate is finite. Stated another way, normal cells have an intrinsically determined limit to the number of cell divisions through which they can proceed. This phenomenon has been termed “replicative cellular senescence” and is an intrinsic anticancer mechanism that limits a cell's proliferative ability, thereby preventing neoplastic transformation. Another form of senescence is “premature cellular senescence.” Premature cellular senescence, like replicative cellular senescence, is a terminal fate of mitotic cells, characterized by permanent cell cycle arrest. Unlike replicative cellular senescence, however, premature cellular senescence does not require telomere deterioration and can be induced by a variety of stressors including, but not limited to, ultraviolet light, reactive oxygen species, chemotherapeutics, environmental toxin, cigarette smoking, ionizing radiation, distortion of chromatin structure, excessive mitogenic signaling, and oncogenic mutations. Still another form of senescence is therapy-induced senescence (TIS) which refers to the phenomenon of a subset of tumor cells being forced into a senescent state by therapeutic agents. TIS is known to develop because of certain treatments, including radiotherapy and chemotherapy.

The number of senescent cells in various organs and tissues of a subject increases with age. The accumulation of senescent cells may drive the deterioration that underlies aging and age-related diseases. For example, the accumulation of senescent cells in aged tissue may contribute to age-associated tissue dysfunction, reduced regenerative capacity, and disease. In this context, senescence is considered deleterious because it contributes to decrements in tissue renewal and function. As a non-limiting example, an aged tissue may lack the ability to respond to stress when proliferation is required thereby resulting in the reduced fitness seen with aging. A key component of this model is that substantial numbers of senescent cells should be present in tissues with aging, without, or prior to, pathology.

### (a) Senescent Cells

A senescent cell may be a cell that ceases to divide but remains metabolically active. The non-dividing cells may remain viable for many weeks, but fail to grow/replicate DNA despite the presence of ample space, nutrients and growth factors in the medium. Thus, the senescence growth arrest is essentially permanent because senescent cells cannot be stimulated to proliferate by known physiological stimuli. Further, a senescent cell of the invention may be resistant to certain apoptotic signals and may acquire widespread changes in gene expression. The resistance to apoptosis may explain the increase in senescent cells with age. Manipulation of pro- and anti-apoptotic proteins may cause cells that are destined to die by apoptosis to senesce and, conversely, cause cells that are destined to senesce to undergo apoptosis.

A senescent cell of the invention may be senescent due to replicative cellular senescence, premature cellular senescence or therapy-induced senescence. Senescent cells that are senescent due to replication may have undergone greater than 60 population doublings. Alternatively, senescent cells that are senescent due to replication may have undergone greater than 40, greater than 50, greater than 60, greater than 70 or greater than 80 population doublings. A senescent cell that is prematurely cellular senescent may be induced by, but not limited to, ultraviolet light, reactive oxygen species, chemotherapeutics, environmental toxin, cigarette smoking, ionizing radiation, distortion of chromatin structure, excessive mitogenic signaling, and oncogenic mutations. In a specific embodiment, premature cellular senescence may be induced by ionizing radiation (IR). In another specific embodiment, premature cellular senescence may also be induced by ectopic transfection with Ras oncogene. A senescent cell that is therapy-induced senescent may have been exposed to DNA-damaging therapy.

A senescent cell of the invention may generally be a eukaryotic cell. Non-limiting examples of senescent cells may include, but are not limited to, mammary epithelial cells, keratinocytes, cardiac myocytes, chondrocytes, endothelial cells (large vessels), endothelial cells (microvascular), epithelial cells, fibroblasts, follicle dermal papilla cells, hepatocytes, melanocytes, osteoblasts, preadipocytes, cells of the immune system, skeletal muscle cells, smooth muscle cells, adipocytes, neurons, glial cells, contractile cells, exocrine secretory epithelial cells, extracellular matrix cells, hormone secreting cells, keratinizing epithelial cells, islet cells, lens cells, mesenchymal stem cells, pancreatic acinar cells, paneth cells of the small intestine, cells of hemopoietic linage, cells of the nervous system, sense organ and peripheral neuron supporting cells and wet stratified barrier epithelial cells.

Further, a senescent cell of the invention may be found in renewable tissues, including the vasculature, hematopoietic system, epithelial organs and the stroma. A senescent cell of the invention may also be found at sites of aging or chronic age-related pathology, such as osteoarthritis and atherosclerosis. Further, a senescent cell of the invention may be associated with benign dysplastic or preneoplastic lesions and benign prostatic hyperplasia. In an embodiment, a senescent cell of the invention may be found in normal and/or tumor tissues following DNA-damaging therapy. In a specific embodiment, a senescent cell may be found at a site of aging or age-related pathology. In another specific embodiment, a senescent cell may be found at a site of senescence-associated pathology.

An age-related pathology may include any disease or condition which is fully or partially mediated by the induction or maintenance of a non-proliferating or senescent state in a cell or a population of cells in a subject. Non-limiting examples include age-related tissue or organ decline which may lack visible indication of pathology, or overt pathology such as a degenerative disease or a function-decreasing disorder. For example, Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis, and others. A method of identifying an age-related disease or condition as described herein may include detecting the presence of senescent cells.

A senescence-associated pathology may include any disease or condition which is fully or partially mediated by the induction or maintenance of a non-proliferating or senescent state in a cell or a population of cells in a subject. Non-limiting examples include cardiovascular diseases such as angina, aortic aneurysm, arrhythmia, brain aneurysm, cardiac diastolic dysfunction, cardiac fibrosis, cardiac stress resistance, cardiomyopathy, carotid artery disease, coronary thrombosis, endocarditis, hypercholesterolemia, hyperlipidemia, mitral valve prolapsed, and peripheral vascular disease; inflammatory or autoimmune diseases such as herniated intervertebral disc, inflammatory bowel disease, kyphosis, oral mucositis, lupus, interstital cystitis, scleroderma, and alopecia; neurodegenerative diseases such as dementia, Huntington's disease, motor neuron dysfunction, age-related memory decline, and depression/mood disorders; metabolic diseases such as diabetic ulcer and metabolic syndrome; pulmonary diseases such as age-related loss of pulmonary function, asthma, bronchiectasis, cystic fibrosis, emphysema, and age-associated sleep apnea; gastrointestinal diseases such as Barrett's esophagus; age-related disorders such as liver fibrosis, muscle fatigue, oral submucosa fibrosis, pancreatic fibrosis, benign prostatic hyperplasia (BPH), and age-related sleep disorders; reproductive disorders such as menopause (male and female), egg supply (female), sperm viability (male), fertility (male and female), sex drive, and erectile function and arousal (male and female); dermatological diseases such as atopic dermatitis, cutaneous lupus, cutaneous lymphomas, dysesthesia, eczema, eczematous eruptions, eosinophilic dermatosis, fibrohistocytic proliferations of skin, hyperpigmentation, immunobullous dermatosis, nevi, pemphigoid, pemphigus, pruritis, psoriasis, rashes, reactive neutrophilic dermatosis, rhytides, and urticarial; and other diseases such as diabetic wound healing, post-transplant kidney fibrosis, and carotid thrombosis. A method of identifying a senescence-associated pathology as described herein may include detecting the presence of senescent cells.

### (b) Detecting Senescent Cells

In an aspect, a method of the invention may comprise detecting senescent cells. Senescent cells may be detected in vivo or in vitro. Suitable markers for detecting senescent cells in vitro and in vivo are known in the art. For example, methods to detect senescent cells may include, but are not limited to, detecting lack of DNA replication by incorporation of a DNA-staining reagent (e.g. 5-bromodeoxyuridine (BrdU), ³H-thymidine), immunostaining for proteins such as proliferating cell nuclear antigen (PCNA) and Ki-67, histochemical staining for senescence-associated β-galactosidase (SA-β-gal), detecting expression of p16, p19, Pail, Igfbp2, IL-6, Mmp13, Nrg1, differentiated embryo-chondrocyte expressed-1 (DEC1), p15 (a CDK1) and decoy death receptor-2 (DCR2), detecting cytological markers such as senescence-associated heterochromatin foci (SAHFs) and senescence-associated DNA-damage foci (SDFs). SAHFs may be detected by the preferential binding of DNA dyes, such as 4′,6-diamidino-2-phenylindole (DAPI), and the presence of certain heterochromatin-associated histone modifications (for example, H3 Lys9 methylation) and proteins (for example, heterochromatin protein-1 (HP1)). Additionally, senescent cells may be detected as described in U.S. Pat. No. 5,491,069 and US Patent Application No. 2010/0086941. In certain embodiments, senescent cells are detected by histochemical staining for SA-β-gal.

In certain embodiments, one or more senescent cells are detected in a sample. A sample may be a cell sample, a tissue sample, or a biopsy sample from a subject. For instance, a sample may be tissue biopsy material. As such, a tissue sample may be from esophagus, stomach, liver, gallbladder, pancreas, adrenal glands, bladder, gallbladder, large intestine, small intestine, kidneys, liver, pancreas, colon, stomach, thymus, spleen, brain, spinal cord, nerves, adipose tissue, heart, lungs, eyes, corneal, skin or islet tissue or organs. Alternatively, a sample may be a cell sample. As such, a cell sample may be oocytes and/or spermatozoa, mesenchymal stem cells, adipocytes, central nervous system neurons and glial cells, contractile cells, exocrine secretory epithelial cells, extracellular matrix cells, hormone secreting cells, keratinizing epithelial cells, islet cells, kidney cells, lens cells, pancreatic acinar cells, paneth cells of small intestine, primary cells of hemopoietic lineage, primary cells of the nervous system, sense organ and peripheral neuron supporting cells or wet stratified barrier epithelial cells. Detection of senescent cells may be used to assess the replicative history of tissues, thereby providing a method for evaluation of the physiological, in contrast to the chronological age of the tissue.

The number of senescent cells may increase with age. The number of senescent cells in a tissue or sample may be from less than 1% to greater than 15%. In an embodiment, the number of senescent cells in a tissue or sample may be less than 1%, less than 2%, less than 3%, less than 4%, or less than 5%. In another embodiment, the number of senescent cells in a tissue or sample may be about 5%, about 6%, about 7%, about 8%, about 9%, or about 10%. In still another embodiment, the number of senescent cells in a tissue or sample may be greater than 10%, greater than 11%, greater than 12%, greater than 13%, greater than 14%, or greater than 15%.

### (C) Measuring Cell Death

In an aspect, a method of the invention may comprise measuring cell death of senescent cells. Methods of measuring cell death are known in the art. For example, cell death may be measured by Giemsa staining, trypan blue exclusion, acridine orange/ethidium bromide (AO/EB) double staining for fluorescence microscopy and flow cytometry, propidium iodide (PI) staining, annexin V assay, TUNEL assay, DNA ladder, LDH activity, and MTT assay. In a preferred embodiment, cell death is due to induction of apoptosis. Cell death due to induction of apoptosis may be measured by observation of morphological characteristics including cell shrinkage, cytoplasmic condensation, chromatin segregation and condensation, membrane blebbing, and the formation of membrane-bound apoptotic bodies. Cell death due to induction of apoptosis may be measured by observation of biochemical hallmarks including internucleosomal DNA cleavage into oligonucleosome-length fragments. Traditional cell-based methods of measuring cell death due to induction of apoptosis include light and electron microscopy, vital dyes, and nuclear stains. Biochemical methods include DNA laddering, lactate dehydrogenase enzyme release, and MTT/XTT enzyme activity. Additionally, terminal deoxynucleotidyl transferase-mediated dUTP-biotin nick end labeling of DNA fragments (TUNEL) and in situ end labeling (ISEL) techniques are used, which when used in conjunction with standard flow cytometric staining methods yield informative data relating cell death to various cellular parameters, including cell cycle and cell phenotype. See Loo and Rillema, Methods Cell Biol. 1998; 57:251-64, which is incorporated herein by reference, for a review of these methods. In an embodiment, cell death due to apoptosis may be measured by the induction of caspase-8. In the absence of FLIP, procaspase-8 dimerization induces full processing and activation of caspase-8, leading to the release of active caspase-8 to the cytosol and activation of apoptosis. In the presence of FLIP, procaspase-8 remains mostly uncleaved and thus non-functional.

The results of these methods may be used to determine the percentage of viable cells. In a preferred embodiment, cell death may be measured as a reduction in viable cells. Since a composition of the invention selectively kills senescent cells, a reduction in viable cells is indicative of a reduction in senescent cells. As described in Section II(b), the number of senescent cells in a sample may be from less than 1% to greater than 15%. As such, a reduction in viable cells following administration of modulator of FLIP of the invention may be greater than 15% to less than 1%. For example, the reduction in viable cells may be less than 1%, less than 2%, less than 3%, less than 4%, or less than 5%. Alternatively, the reduction in viable cells may be about 5%, about 6%, about 7%, about 8%, about 9%, or about 10%. Additionally, the reduction in viable cells may be greater than 10%, greater than 11%, greater than 12%, greater than 13%, greater than 14%, or greater than 15%.

### (d) Administration

In certain aspects, a therapeutically effective amount of a composition of the invention may be administered to a subject. Administration is performed using standard effective techniques, including peripherally (i.e. not by administration into the central nervous system) or locally to the central nervous system. Peripheral administration includes but is not limited to intravenous, intraperitoneal, subcutaneous, pulmonary, transdermal, intramuscular, intranasal, buccal, sublingual, or suppository administration. Local administration, including directly into the central nervous system (CNS) includes but is not limited to via a lumbar, intraventricular or intraparenchymal catheter or using a surgically implanted controlled release formulation.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

For therapeutic applications, a therapeutically effective amount of a composition of the invention is administered to a subject. A “therapeutically effective amount” is an amount of the therapeutic composition sufficient to produce a measurable response (e.g., cell death of senescent cells, an anti-aging response, an improvement in symptoms associated with a degenerative disease, or an improvement in symptoms associated with a function-decreasing disorder). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, age, the age-related disease or condition, the degenerative disease, the function-decreasing disorder, the symptoms, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

The frequency of dosing may be daily or once, twice, three times or more per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Typical dosage levels can be determined and optimized using standard clinical techniques and will be dependent on the mode of administration.

### (e) Subject

A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

The human subject may be of any age. However, since senescent cells are normally associated with aging, a human subject may be an older human subject. In some embodiments, the human subject may be about 30, 35, 40, 45, 50, 55, 60, 65, 70, 75, 80, 85, 90, 95 years of age or older. In some preferred embodiments, the human subject is 30 years of age or older. In other preferred embodiments, the human subject is 40 years of age or older. In other preferred embodiments, the human subject is 45 years of age or older. In yet other preferred embodiments, the human subject is 50 years of age or older. In still other preferred embodiments, the human subject is 55 years of age or older. In other preferred embodiments, the human subject is 60 years of age or older. In yet other preferred embodiments, the human subject is 65 years of age or older. In still other preferred embodiments, the human subject is 70 years of age or older. In other preferred embodiments, the human subject is 75 years of age or older. In still other preferred embodiments, the human subject is 80 years of age or older. In yet other preferred embodiments, the human subject is 85 years of age or older. In still other preferred embodiments, the human subject is 90 years of age or older.

Additionally, a subject in need thereof may be a subject suffering from an age-related disease or condition as described below.

### (f) Aging and Age-Related Diseases

It has been demonstrated that senescent cells drive age-related pathologies and that selective elimination of these cells can prevent or delay age-related deterioration. Thus, senescent cells may be therapeutic targets in the treatment of aging and age-related disease. As such, removal of senescent cells may delay tissue dysfunction and extend health span. Clearance of senescent cells is expected to improve tissue milieu, thereby improving the function of the remaining non-senescent cells.

The present disclosure provides a method for delaying at least one feature of aging in a subject, the method comprising administering a composition comprising a therapeutically effective amount of a compound that modulates FLIP to a subject. As used herein, “a feature of aging” may include, but is not limited to, systemic decline of the immune system, muscle atrophy and decreased muscle strength, decreased skin elasticity, delayed wound healing, retinal atrophy, reduced lens transparency, reduced hearing, osteoporosis, sarcopenia, hair graying, skin wrinkling, poor vision, frailty, and cognitive impairment.

In an aspect, a composition of in the invention selectively kills senescent cells. In this way, targeting senescent cells during the course of aging may be a preventative strategy. Accordingly, administration of a composition comprising a therapeutically effective amount of a compound that modulates FLIP to a subject may prevent comorbidity and delay mortality in an older subject. Further, selective killing of senescent cells may boost the immune system, extend the health span, and improve the quality of life in a subject. Additionally, selective killing of senescent cells may delay sarcopenia. Sarcopenia is the degenerative loss of skeletal muscle mass, quality, and strength associated with aging. As such, a delay in sarcopenia may reduce frailty, reduce risk of falling, reduce fractures, and reduce functional disability in a subject. Furthermore, selective killing of senescent cells may delay aging of the skin. Aged skin has increased wrinkles, decreased immune barrier function and increased susceptibility to skin cancer and trauma. As such, selective killing of senescent cells may delay skin wrinkling, delay the onset of decreased immune barrier function and decrease susceptibility to skin cancer and trauma in a subject. Selective killing of senescent cells may also delay the onset of retinal atrophy and reduced lens transparency as measured by vision tests.

Methods of measuring aging are known in the art. For example, aging may be measured in the bone by incident non-vertebral fractures, incident hip fractures, incident total fractures, incident vertebral fractures, incident repeat fractures, functional recovery after fracture, bone mineral density decrease at the lumbar spine and hip, rate of knee buckling, NSAID use, number of joints with pain, and osteoarthritis. Aging may also be measured in the muscle by functional decline, rate of falls, reaction time and grip strength, muscle mass decrease at upper and lower extremities, and dual tasking 10-meter gait speed. Further, aging may be measured in the cardiovascular system by systolic and diastolic blood pressure change, incident hypertension, major cardiovascular events such as myocardial infarction, stroke, congestive heart disease, and cardiovascular mortality. Additionally, aging may be measured in the brain by cognitive decline, incident depression, and incident dementia. Also, aging may be measured in the immune system by rate of infection, rate of upper respiratory infections, rate of flu-like illness, incident severe infections that lead to hospital admission, incident cancer, rate of implant infections, and rate of gastrointestinal infections. Other indications of aging may include, but not limited to, decline in oral health, tooth loss, rate of GI symptoms, change in fasting glucose and/or insulin levels, body composition, decline in kidney function, quality of life, incident disability regarding activities of daily living, and incident nursing home admission. Methods of measuring skin aging are known in the art and may include trans-epidermal water loss (TEWL), skin hydration, skin elasticity, area ratio analysis of crow's feet, sensitivity, radiance, roughness, spots, laxity, skin tone homogeneity, softness, and relief (variations in depth).

The present disclosure also provides a method of treating an age-related disease or condition, the method comprising administering a composition comprising a therapeutically effective amount of a compound that modulates FLIP to a subject in need thereof, provided the age-related disease or condition is not cancer. As used herein, “age-related disease or condition” may include, but is not limited to, a degenerative disease or a function-decreasing disorder such as Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, cancer treatment-related disability such as atrophy and fibrosis in various tissues, brain and heart injury, and therapy-related myelodysplastic syndromes, and diseases associated with accelerated aging and/or defects in DNA damage repair and telomere maintenance such as Hutchinson-Gilford progeria syndrome, Werner syndrome, Cockayne syndrome, exroderma pigmentosum, ataxia telangiectasia, Fanconi anemia, dyskeratosis congenital, aplastic anemia, idiopathic pulmonary fibrosis. Methods of diagnosing and identifying an age-related disease or condition are known in the art.

The present disclosure also provides a method of treating a senescence-associated disease or condition, the method comprising administering a composition comprising a therapeutically effective amount of a compound that modulates FLIP to a subject in need thereof, provided the senescence-associated disease or condition is not cancer. As used herein, “senescence-associated disease or condition” may include, but is not limited to, cardiovascular diseases such as angina, aortic aneurysm, arrhythmia, brain aneurysm, cardiac diastolic dysfunction, cardiac fibrosis, cardiac stress resistance, cardiomyopathy, carotid artery disease, coronary thrombosis, endocarditis, hypercholesterolemia, hyperlipidemia, mitral valve prolapsed, and peripheral vascular disease; inflammatory or autoimmune diseases such as herniated intervertebral disc, inflammatory bowel disease, kyphosis, oral mucositis, lupus, interstitial cystitis, scleroderma, and alopecia; neurodegenerative diseases such as dementia, Huntington's disease, motor neuron dysfunction, age-related memory decline, and depression/mood disorders; metabolic diseases such as diabetic ulcer and metabolic syndrome; pulmonary diseases such as age-related loss of pulmonary function, asthma, bronchiectasis, cystic fibrosis, emphysema, and age-associated sleep apnea; gastrointestinal diseases such as Barrett's esophagus; age-related disorders such as liver fibrosis, muscle fatigue, oral submucosa fibrosis, pancreatic fibrosis, benign prostatic hyperplasia (BPH), and age-related sleep disorders; reproductive disorders such as menopause (male and female), egg supply (female), sperm viability (male), fertility (male and female), sex drive, and erectile function and arousal (male and female); dermatological diseases such as atopic dermatitis, cutaneous lupus, cutaneous lymphomas, dysesthesia, eczema, eczematous eruptions, eosinophilic dermatosis, fibrohistocytic proliferations of skin, hyperpigmentation, immunobullous dermatosis, nevi, pemphigoid, pemphigus, pruritis, psoriasis, rashes, reactive neutrophilic dermatosis, rhytides, and urticarial; and other diseases such as diabetic wound healing, post-transplant kidney fibrosis, and carotid thrombosis. Methods of diagnosing and identifying a senescence-associated disease or condition are known in the art.

The present disclosure also provides a method of killing therapy-induced senescent cells. The method comprises administering a composition comprising a therapeutically effective amount of a compound that modulates FLIP to a subject that has received DNA-damaging therapy and killing therapy induced-senescent cells in normal and tumor tissues following DNA-damaging therapy.

Non-limiting examples of DNA-damaging therapy may include γ-irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine. clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, mitoxantrone, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine) and estramustine.

Based on the observation that ionizing radiation and various chemotherapeutic agents elicit a marked senescence response in vivo, therapy-induced senescent cells may be a cause of long-term ramifications after DNA-damaging therapy, such as cancer therapy. As such, the systemic accumulation of therapy-induced senescent cells may drive accelerated physical decline in cancer survivors. Accelerated physical decline may also be referred to as accelerated aging. Accordingly, once a tumor is removed by systemic radiation or chemotherapy, senescence may be triggered in a variety of other organs, leading to long-term ramifications for the patient. Long-term ramifications may include reduced quality of life predisposing the subject to disabilities and comorbidities. For example, a subject that has received DNA-damaging therapy may experience a disproportionate decline in physical function, such as inability to walk up stairs or to reach up to put things onto shelves and/or increased functional disabilities such as difficulty, eating, dressing and maintaining adequate hygiene. These long-term ramifications provide a link between accelerated aging and cancer treatment. A method to measure accelerated aging may be as described in methods of measuring aging as above. Accordingly, administration of a composition comprising an inhibitor of the invention to a subject may prevent accelerated aging in a subject who has received DNA damaging therapy.

In any of the foregoing embodiments, a composition of the disclosure may also be administered in combination with one or more additional drug or therapeutically active agent. For example, a composition comprising one or more compounds that interact with FAS and/or DRs and induce apoptosis and/or Bcl-2 inhibitors may also be administered to the subject. Specifically, a composition comprising one or more compounds that interact with FAS and/or DRs and induce apoptosis as described in U.S. 62/106,573 and/or Bcl-2 inhibitors as described in PCT/US2015/029208, the disclosures of which are hereby incorporated by reference in their entirety, may also be administered to the subject. Still further, a composition comprising one or more piperlongumines or derivatives thereof may also be administered to the subject. Specifically, a composition comprising one or more piperlongumines or derivatives thereof as described in PCT/US2015/041470, the disclosure of which is hereby incorporated by reference in its entirety, may also be administered to the subject.

### (g) Screening Assays

The invention provides a method (also referred to herein as a “screening assay”) for identifying modulators, i.e., candidate or test compounds or agents (e.g., peptides, peptidomimetics, small molecules or other drugs) which downregulate FLIP, for example, FLIP nucleic acid expression, FLIP protein expression or FLIP activity. A modulator of FLIP may also be referred to as a senolytic drug. A modulator of FLIP may directly or indirectly downregulate FLIP.

Screening assays may also be used to identify molecules that modulate FLIP nucleic acid expression, FLIP protein expression or FLIP activity. For example, FLIP inhibits the activation of caspase 8 to prevent apoptosis. Accordingly, apoptosis, cell viability, or caspase-8 may be measured as an indication of FLIP activity or expression. Apoptosis and cell viability may be measured using methods standard in the art as described above in Section II(c). Caspase-8 may be measured using methods to detect protein expression as described in Section I. Alternatively, FLIP nucleic acid expression or protein expression may be measured as an indication of FLIP activity or expression. Methods to detect FLIP nucleic acid or protein expression are standard in the art. For instance, see Section I and/or the Examples.

In one embodiment, the invention provides assays for screening candidate or test compounds which bind to or modulate the activity or expression of FLIP. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145). Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or on phage (Scott and Smith (1990) Science 249:386-390; Devlin (1990) Science 249:404-406; Cwirla et al. (1990) Proc. Natl. Acad. Sci. 87:6378-6382; and Felici (1991) J. Mol. Biol. 222:301-310).

In one embodiment, an assay is one in which cells are contacted with a test compound and the ability of the test compound to downregulate FLIP is determined. Determining the ability of the test compound to downregulate FLIP may be accomplished, for example, by detecting FLIP protein expression. Numerous methods for detecting protein are known in the art and are contemplated according to the invention, see Section I. Specifically, an immunoblot may be used to detect FLIP protein. Alternatively, determining the ability of the test compound to downregulate FLIP may be accomplished, for example, by measuring cell death or apoptosis. Methods of measuring cell death or apoptosis are known in the art, see Section II(c). Another method for determining the ability of the test compound to downregulate FLIP may be accomplished by a reporter assay for FLIP expression. For example, FLIP protein expression may be fused to a reporter protein such that FLIP expression may be monitored by measuring the expression of the reporter protein. By way of example, reporter proteins may include a fluorescent protein, luciferase, alkaline phosphatase, beta-galactosidase, beta-lactamase, horseradish peroxidase, and variants thereof. In another embodiment, determining the ability of the test compound to downregulate FLIP may be accomplished, for example, by detecting FLIP nucleic acid expression. Methods of measuring nucleic acid expression are known in the art, see Section I. Specifically, FLIP mRNA may be detected via standard methods. Determining the ability of the test compound to downregulate FLIP may be accomplished, for example, by determining the ability of FLIP to inhibit the activation of caspase 8. Methods for detecting caspase 8 are known in the art. For example, methods to detect protein expression may be used to detect caspase 8.

In another embodiment, an assay is one in which FLIP is contacted with a test compound and the ability of the test compound to bind to FLIP is determined. Determining the ability of the test compound to bind to FLIP may be accomplished, for example, by coupling the test compound with a radioisotope or enzymatic label such that binding of the test compound to FLIP may be determined by detecting the labeled compound in a complex. For example, test compounds can be labeled with ¹²⁵I, ³⁵S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product.

In yet another embodiment, an assay of the present invention is a cell-free assay comprising contacting FLIP with a test compound and determining the ability of the test compound to bind to FLIP. Binding of the test compound to FLIP may be determined either directly or indirectly. In one embodiment, a competitive binding assay includes contacting FLIP with a compound known to bind FLIP to form an assay mixture, contacting the assay mixture with a test compound, and determining the ability of the test compound to interact with FLIP, wherein determining the ability of the test compound to interact with FLIP comprises determining the ability of the test compound to preferentially bind to FLIP as compared to the known binding compound.

In another embodiment, an assay is a cell-free assay comprising contacting FLIP with a test compound and determining the ability of the test compound to modulate (e.g., stimulate or inhibit) the activity of FLIP. Determining the ability of the test compound to modulate the activity of FLIP can be accomplished, for example, by determining the ability of FLIP to bind to or interact with a FLIP target molecule. In an alternative embodiment, determining the ability of the test compound to modulate the activity of FLIP can be accomplished by determining the ability of FLIP to further modulate a FLIP target molecule. As used herein, a “target molecule” is a molecule with which FLIP binds or interacts in nature. Exemplary target molecules include initiator caspases such as procaspase-8 and procaspase-10, adaptor proteins such as FADD and TRADD, and other proteins known to interact with a death effector domain (DED).

In another embodiment, modulators of FLIP expression are identified in a method in which a cell is contacted with a candidate compound and the expression of the FLIP promoter, mRNA or protein in the cell is determined. The level of expression of FLIP mRNA or protein in the presence of the candidate compound is compared to the level of expression of FLIP mRNA or protein in the absence of the candidate compound. The candidate compound can then be identified as a modulator of FLIP expression based on this comparison. For example, when expression of FLIP mRNA or protein is greater (statistically significantly greater) in the presence of the candidate compound than in its absence, the candidate compound is identified as a stimulator of FLIP mRNA or protein expression. Alternatively, when expression of FLIP mRNA or protein is less (statistically significantly less) in the presence of the candidate compound than in its absence, the candidate compound is identified as an inhibitor of FLIP mRNA or protein expression. The level of FLIP mRNA or protein expression in the cells can be determined by methods described herein for detecting FLIP mRNA or protein. The activity of the FLIP promoter can be assayed by linking the FLIP promoter to a reporter gene such as luciferase, secreted alkaline phosphatase, or beta-galactosidase and introducing the resulting construct into an appropriate vector, transfecting a host cell line, and measuring the activity of the reporter gene in response to test compounds.

This invention further pertains to novel agents identified by the above-described screening assays and uses thereof for treatments as described herein.

### (h) Predictive Medicine

The present invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the present invention relates to diagnostic assays for determining FLIP protein and/or nucleic acid expression as well as FLIP activity, in the context of a biological sample (e.g., blood, serum, cells, tissue) to thereby determine whether an individual is afflicted with a disease or disorder, or is at risk of developing a disorder, associated with senescent cells. Accordingly, the invention also provides for prognostic (or predictive) assays for determining whether an individual is at risk of developing a disorder associated with aging or age-related diseases. For example, FLIP protein, nucleic acid expression or activity can be assayed in a biological sample. Such assays can be used for prognostic or predictive purpose to thereby prophylactically treat an individual prior to the onset of a disorder characterized by senescent cells.

Another aspect of the invention provides methods for determining FLIP protein, nucleic acid expression or FLIP activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs or other compounds) on the expression or activity of FLIP in clinical trials.

These and other agents are described in further detail in the following sections.

i. Diagnostic Assays

An exemplary method for detecting the presence or absence of FLIP in a sample involves obtaining a sample from a test subject and contacting the sample with a compound or an agent capable of detecting FLIP protein or nucleic acid (e.g., mRNA, genomic DNA) such that the presence of FLIP is detected in the sample. An agent for detecting FLIP mRNA or genomic DNA is a labeled nucleic acid probe capable of hybridizing to FLIP mRNA or genomic DNA. The nucleic acid probe can be, for example, a full-length FLIP nucleic acid or a portion thereof, such as an oligonucleotide of at least 15, 30, 50, 100, 250, 500, 750 or more nucleotides in length and sufficient to specifically hybridize under stringent conditions to mRNA or genomic DNA. Other suitable probes for use in the diagnostic assays of the invention are described herein.

An agent for detecting FLIP protein can be an antibody capable of binding to FLIP protein, preferably an antibody with a detectable label. Antibodies can be polyclonal, or more preferably, monoclonal. An intact antibody, or a fragment thereof (e.g., Fab or F(ab′)2) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The detection method of the invention can be used to detect FLIP mRNA, protein, or genomic DNA in a sample in vitro as well as in vivo. For example, in vitro techniques for detection of FLIP mRNA include Northern hybridizations and in situ hybridizations. In vitro techniques for detection of FLIP protein include enzyme linked immunosorbent assays (ELISAs), Western blots, immunoprecipitations and immunofluorescence. In vitro techniques for detection of FLIP genomic DNA include Southern hybridizations. Furthermore, in vivo techniques for detection of FLIP protein include introducing into a subject a labeled anti-FLIP antibody. For example, the antibody can be labeled with a radioactive marker whose presence and location in a subject can be detected by standard imaging techniques.

In one embodiment, the sample contains protein molecules from the test subject. Alternatively, the biological sample can contain mRNA molecules from the test subject or genomic DNA molecules from the test subject.

In another embodiment, the methods further involve obtaining a control sample from a control subject, contacting the control sample with a compound or agent capable of detecting FLIP protein, mRNA, or genomic DNA, such that the presence of FLIP protein, mRNA or genomic DNA is detected in the sample, and comparing the presence of FLIP protein, mRNA or genomic DNA in the control sample with the presence of FLIP protein, mRNA or genomic DNA in the test sample.

The invention also encompasses kits for detecting the presence of FLIP in a sample. The kit may comprise a labeled compound or agent capable of detecting FLIP protein or mRNA in a biological sample and means for determining the amount of FLIP in the sample.

For antibody-based kits, the kit may comprise, for example: (1) a first antibody (e.g., attached to a solid support) which binds to FLIP protein; and, optionally, (2) a second, different antibody which binds to FLIP protein or the first antibody and is conjugated to a detectable agent. For oligonucleotide-based kits, the kit may comprise, for example: (1) an oligonucleotide, (e.g., a detectably labeled oligonucleotide), which hybridizes to a FLIP nucleic acid sequence or (2) a pair of primers useful for amplifying a FLIP nucleic acid molecule. The kit may also comprise, a buffering agent, a preservative, or a protein stabilizing agent. The kit may also comprise components necessary for detecting the detectable agent (e.g., an enzyme or a substrate). The kit may also contain a control sample or a series of control samples which can be assayed and compared to the test sample contained. Each component of the kit is usually enclosed within an individual container and all of the various containers are within a single package along with instructions for use.

ii. Prognostic Assays

The methods described herein can furthermore be utilized as diagnostic or prognostic assays to identify subjects having or at risk of developing a disease or disorder associated with senescent cells. For example, the assays described herein, such as the preceding diagnostic assays or the following assays, may be utilized to identify a subject having or at risk of developing an age-related disease. Alternatively, the prognostic assays may be utilized to identify a subject having or at risk for developing such a disease or disorder. Thus, the present invention provides a method in which a test sample is obtained from a subject and FLIP protein or nucleic acid (e.g., mRNA, genomic DNA) is detected, wherein the presence of FLIP protein or nucleic acid is diagnostic for a subject having senescent cells or at risk of developing a disease or disorder associated with senescent cells. As used herein, a “test sample” refers to a biological sample obtained from a subject of interest. For example, a test sample can be a biological fluid (e.g., serum), cell sample, or tissue. Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with senescent cells. Exemplary diseases include, without limitation, aging or age-related diseases as described above.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness associated with aging or age-related diseases.

iii. Monitoring of Effects During Therapeutic Treatment

Monitoring the influence of agents (e.g., drugs, compounds) on the expression or activity of FLIP can be applied not only in basic drug screening, but also in therapeutic treatments. For example, the effectiveness of an agent determined by a screening assay as described herein to downregulate FLIP nucleic acid expression, protein levels, or FLIP activity, can be monitored in subjects. Alternatively, the effectiveness of an agent determined by a screening assay to downregulate FLIP nucleic acid expression, protein levels, or FLIP activity, can be monitored in clinical trials of subjects. In such clinical trials, the expression or activity of FLIP can be used as a “read out”.

For example, and not by way of limitation, treatment with an agent (e.g., compound, drug or small molecule) which modulates FLIP activity (e.g., identified in a screening assay as described herein) can be identified. Thus, to study the effect of agents on aging or age-related diseases, for example, in a clinical trial, cells can be isolated and RNA prepared and analyzed for the levels of expression of FLIP. The levels of FLIP expression (i.e., a gene expression pattern) can be quantified by Northern blot analysis or RT-PCR, as described herein, or alternatively by measuring the amount of protein produced, by one of the methods as described herein, or by measuring the levels of activity of FLIP or other genes. In this way, the FLIP expression pattern can serve as a marker, indicative of the physiological response of the cells to the agent. Accordingly, this response state may be determined before, and at various points during, treatment of the individual with the agent.

In an embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of expression of a FLIP protein, mRNA, or genomic DNA in the preadministration sample; (iii) obtaining one or more post-administration samples from the subject; (iv) detecting the level of expression or activity of the FLIP protein, mRNA, or genomic DNA in the post-administration samples; (v) comparing the level of expression or activity of the FLIP protein, mRNA, or genomic DNA in the pre-administration sample with the FLIP protein, mRNA, or genomic DNA in the post administration sample or samples; and (vi) altering the administration of the agent to the subject accordingly. For example, increased administration of the agent may be desirable to decrease the expression or activity of FLIP to lower levels than detected, i.e., to increase the effectiveness of the agent. Alternatively, decreased administration of the agent may be desirable to increase the expression or activity of FLIP to higher levels than detected, i.e., to decrease the effectiveness of the agent.

iv. Transcriptional Profiling

The FLIP nucleic acid molecules described herein, including small oligonucleotides, can be used in transcriptionally profiling. For example, these nucleic acids can be used to examine the expression of FLIP in normal tissue or cells and in tissue or cells subject to a disease state, e.g., tissue or cells derived from a patient having a disease of interest or cultured cells which model or reflect a disease state of interest, e.g., senescent cells. By measuring expression of FLIP, together or individually, a profile of expression in normal and disease states can be developed. This profile can be used diagnostically and to examine the effectiveness of a therapeutic regime.

### EXAMPLES

The following examples illustrate various iterations of the invention. However, those of skill in the art should, in light of the present disclosure, appreciate that many changes can be made in the specific embodiments which are disclosed and still obtain a like or similar result without departing from the spirit and scope of the invention.

### Example 1. Senescent Cells Express Increased Levels of FLIP and Fas

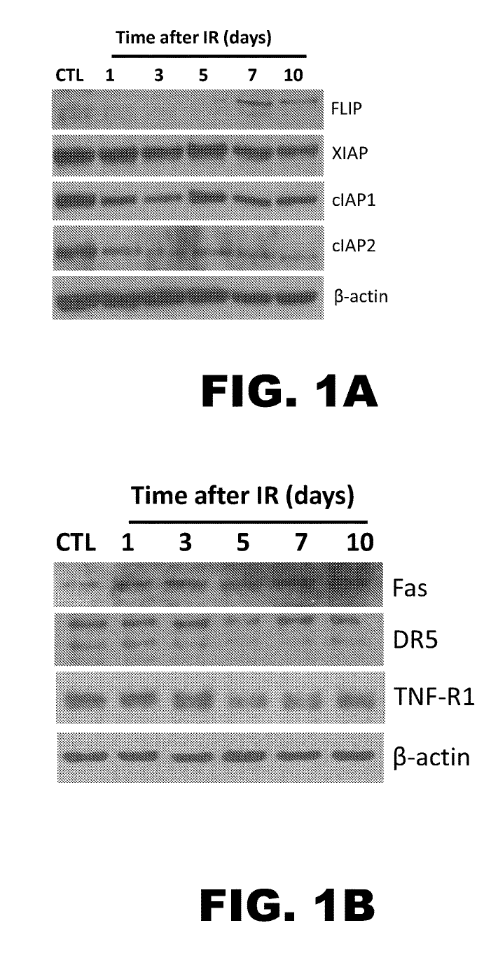

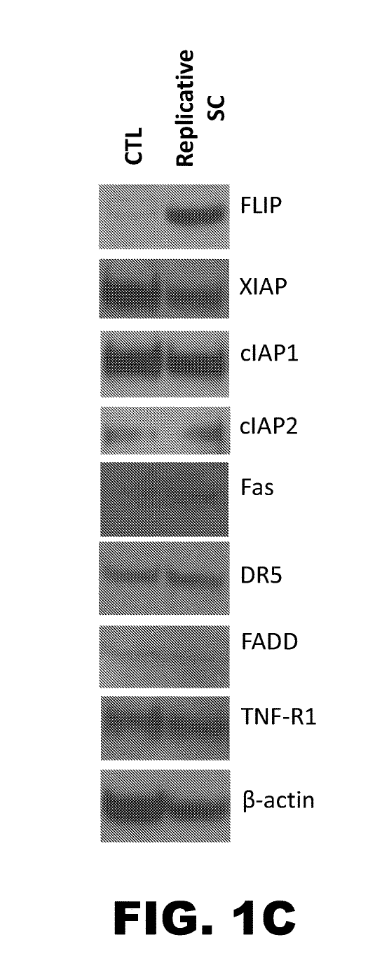

Expression of FLIP, XIAP, cIAP1, cIAP2 and β-actin was analyzed by Western blots in control (CTL) and WI38 human fibroblast cells 1, 3, 5, 7 and 10 days after exposure to 10 Gy γ-irradiation (FIG. 1A). Expression of Fas, DR5, TNF-R1 and β-actin was analyzed by Western blots in control (CTL) and WI38 human fibroblast cells 1, 3, 5, 7 and 10 days after exposure to 10 Gy γ-irradiation (FIG. 1B). Expression of FLIP, XIAP, cIAP1, cIAP2, Fas, DR5, FADD, TNF-R1 and β-actin was analyzed by Western blots in control (CTL) and replicative senescent WI38 human fibroblast cells. The results showed that both IR-induced and replicative senescent cells (SC) expressed increased levels of FLIP and Fas as compared with control cells (FIG. 1C).

### Example 2. Knockdown FLIP Expression by FLIP-shRNA Selectively Kills Senescent Cells

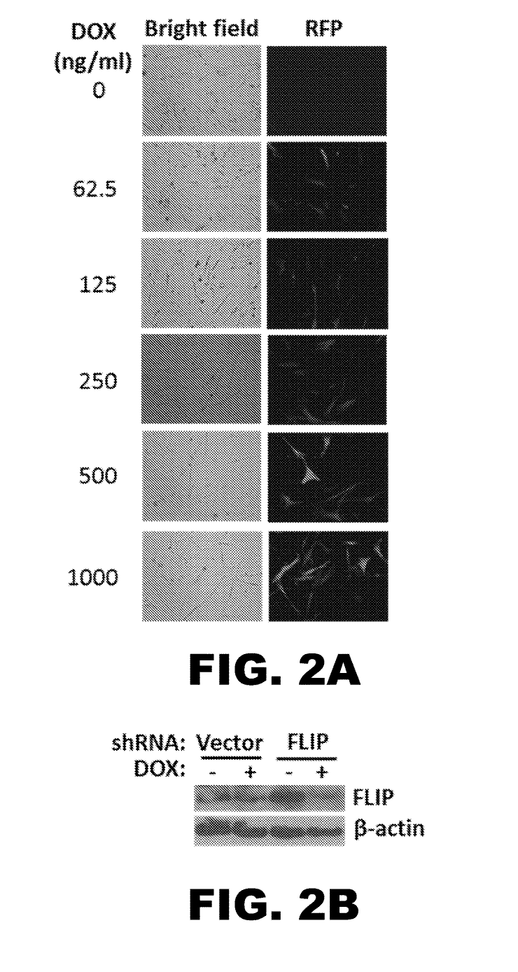

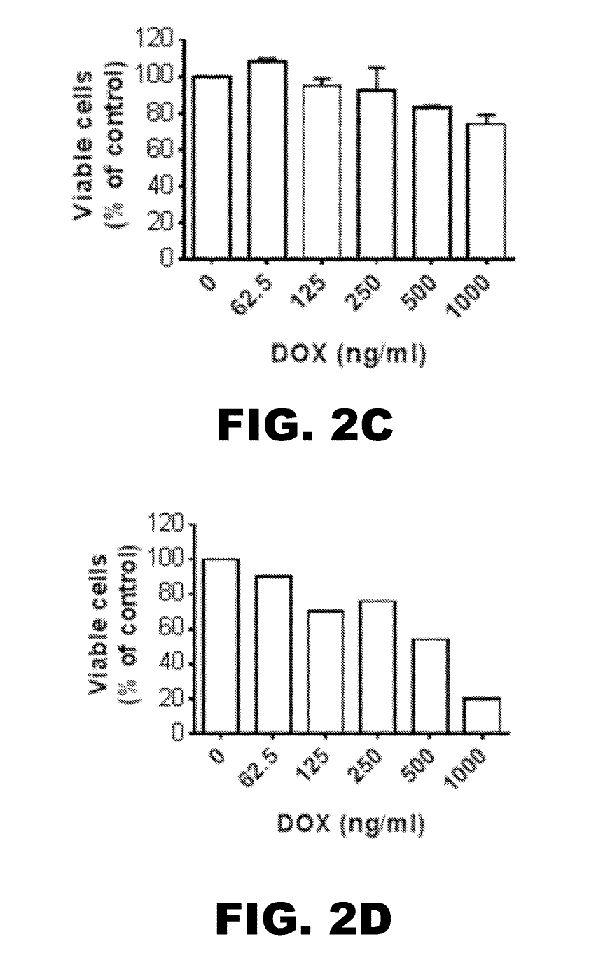

Treatment with doxycycoline (DOX) dose-dependently induces FLIP-shRNA expression in WI38 cell line after the cells were stably transfected with a plasmid containing FLIP-shRNA and red fluorescent protein (RFP) genes (FIG. 2A). Induction of FLIP-shRNA by doxycycoline down-regulates FLIP expression in IR-induced senescent WI38 cells stably transfected with FLIP-shRNA but not in vector transfected cells (FIG. 2B). Induction of FLIP-shRNA expression by DOX selectively kills IR-induced senescent cells (SC) in a DOX-dose-dependent manner but has minimal effect on normal cells (NC) (FIG. 2C,D).

### Example 3. Down-Regulation of FLIP Expression by a Small Molecule Selectively Kills Senescent Cells

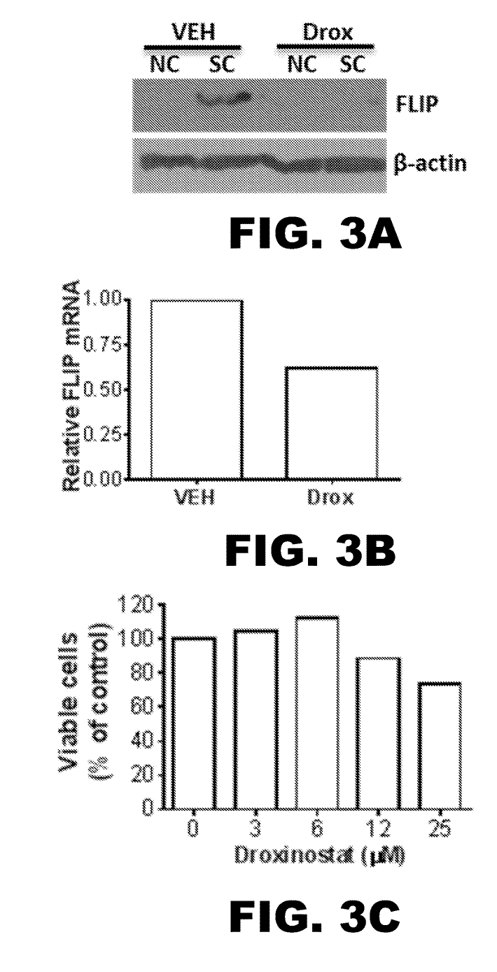

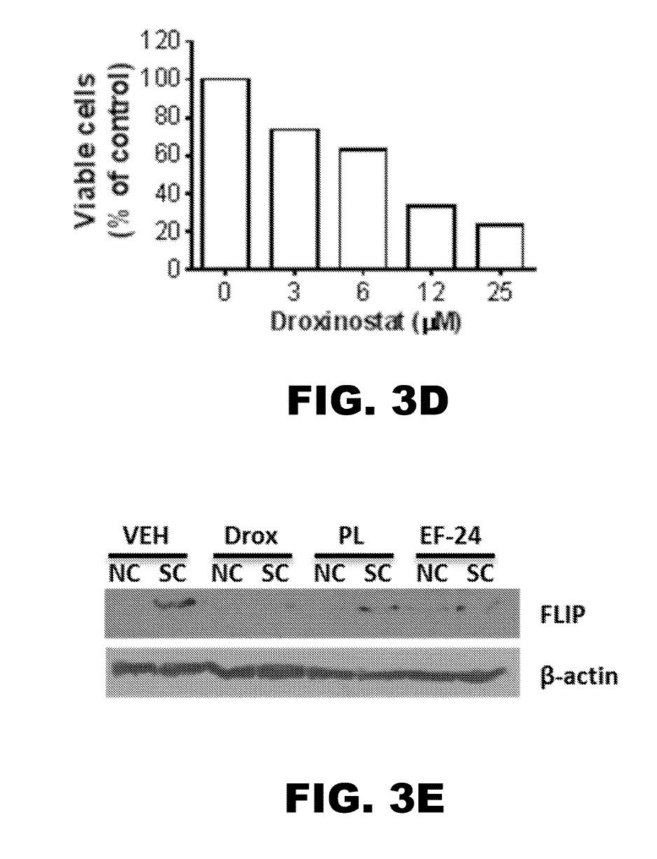

IR-induced senescent (SC) WI38 cells exhibit an increased expression of FLIP, which was down-regulated after treatment with droxinostat (Drox) (FIG. 3A). The expression of FLIP in normal (NC) and IR-induced senescent (SC) WI38 cells was determined by Western blot 24 h after they were treated with vehicle (VEH) or Drox (10 μM). Treatment with droxinostat (Drox) down-regulates the expression of FLIP mRNA in IR-induced senescent (SC) WI38 cells (FIG. 3B). IR-induced senescent (SC) WI38 cells were treated with vehicle (VEH) or Drox (10 μM) for 6 h and the expression of FLIP mRNA in these cells was determined by qPCR. Droxinostat (Drox) selectively kills IR-induced senescent (SC) WI38 cells but has minimal effect on normal (NC) WI38 cells (FIG. 3C,D). Viable cells were determined 72 h after normal (NC) and IR-induced senescent (SC) WI38 cells were treated with vehicle (VEH) or increasing concentrations of Drox. The data are presented as a percentage of control cells treated with VEH. Treatment with droxinostat (Drox), piperlongumine (PL) and EF-24 down-regulates the expression of FLIP in IR-induced senescent (SC) WI38 cells (FIG. 3E). Normal (NC) and IR-induced senescent (SC) WI38 cells were treated with vehicle (VEH), Drox (10 μM), PL (10 μM), and EF-24 (10 μM) for 24 h and the expression of FLIP in these cells was determined by Western blots. Droxinostat (Drox), piperlongumine (PL), and EF-24 selectively kill IR-induced senescent (SC) WI38 cells (Table 1). Viable cells were determined 72 h after normal (NC) and IR-induced senescent (SC) WI38 cells were treated with vehicle (VEH) or increasing concentrations of Drox, PL, and EF-24. LD50 values for these molecules against NC and SC were calculated and presented.

TABLE 1
LD₅₀ value against normal (NC) and senescent (SC) WI38 cells

| | LD₅₀ (μM) | LD₅₀ (μM) | LD₅₀ (μM) |
|---|---|---|---|
| Tested agents | (NC) | (SC) | (N/S) |
| Droxinostat (Drox) | 39.58 | 8.26 | 4.79 |
| Piperlongumine (PL) | 24.25 | 7.05 | 3.44 |
| EF-24 | 6.08 | 1.05 | 5.8 |

### REFERENCES FOR THE EXAMPLES

1. Schimmer A D et al. Cancer Res. 2006; 66:2367-75.
2. Mawji I A et al. Cancer Res. 2007; 67:8307-15.
3. Shirley S & Micheau O. Cancer Letter 2013:332:141-50.
4. Sanders Y Y et al. Redox Biol. 2013; 1:8-16.
5. Safa A R & Pollok K E. Cancer 2011; 3:1639-71.
6. Raja S M et al. Mol Cancer Ther. 2008; 7:2212-23.
7. Lee S-J et al. Int J Oncol. 2011; 38:485-492.
8. Siegelin M D et al. Neuroscie Lett. 2009; 453:92-7.
9. Chen S et al. Cancer Res. 2011; 71:6270-81.

1.-23. (canceled)

24. A method of selectively killing a senescent cell, the method comprising downregulating expression of c-Fas-associated death domain-like interleukin-1 converting enzyme-like inhibitory protein (FLIP) using an agent that has an LD50 in senescent cells that is at least 3 times lower than the agent's LD50 in non-senescent cells
wherein the senescent cell is characterized as expressing p16 and senescence-associated β-galactosidase.

25. The method of claim 24, wherein the agent is droxinostat.

26. The method of claim 24, wherein the agent is piperlongumine.

27. The method of claim 24, wherein the agent is EF24 ((3E,5E)-3,5-bis[(2-fluorophenyl)methylene]-4-piperidinone).

28. The method of claim 24, wherein the agent is a short hairpin RNA (shRNA) specific for FLIP.

29. A method of treating an age related condition in a subject in need thereof, comprising selectively downregulating expression of FLIP in senescent cells according to the method of claim 1, the senescent cells being contained in a tissue in the subject that is affected by the condition.

30. A screening method for identifying a senolytic drug that kills senescent cells with an LD50 that is at least 3 times lower than in non-senescent cells; the method comprising:
(a) contacting senescent cells with a test compound;
(b) measuring expression of c-Fas-associated death domain-like interleukin-1 converting enzyme-like inhibitory protein (FLIP) by the cells contacted with the test compound in step (a);
(c) contacting normal (non-senescent) cells with the same test compound;
(d) measuring expression of FLIP in the cells contacted with the test compound in step (c); and then
(e) identifying the compound as a senolytic drug if the expression of FLIP is downregulated in the senescent cells but has a minimal effect on the non-senescent cells.

31. A method of preparing a pharmaceutical product, comprising:
(1) obtaining a supply of a compound that has been identified as causing down-regulation of FLIP and qualifies as a senolytic drug with sufficient LD50 in senescent cells according to the method of claim 30;
(2) preparing a pharmaceutical composition in which the compound is combined with at least one pharmaceutically acceptable excipient; and
(3) packaging the pharmaceutical composition with instructions for killing senescent cells or treating an age-related condition.

32. The method of claim 30, wherein the test compound is selected from droxinostat, piperlongumine, EF24 ((3E,5E)-3,5-bis[(2-fluorophenyl)methylene]-4-piperidinone), shRNA, and derivatives thereof.

33. The method of claim 30, wherein the expression of FLIP is measured at the mRNA level.

34. The method of claim 30, further comprising determining and comparing LD50 of the test compound in the senescent cells and in the non-senescent cells.

35. The method of claim 7, wherein the senescent cells and the non-senescent cells are obtained from the same cell line, the senescent cells having been treated to induce senescence.

36. A method of treating an age-related condition in a subject in need thereof, comprising:
(a) identifying a senolytic drug that selectively downregulates expression of FLIP in senescent cells, thereby killing the senescent cells with an LD50 that is at least 3 times lower than the drug's LD50 in non-senescent cells; and
(b) administering a pharmaceutical composition containing an amount of the senolytic drug identified instep (a) to the subject, wherein the amount of the drug in the composition and formulation of the composition are effective to alleviate a symptom of the age-related disease in the subject.

37. The method of claim 36, wherein the senolytic drug is selected from droxinostat, piperlongumine, EF24 ((3E,5E)-3,5-bis[(2-fluorophenyl)methylene]-4-piperidinone), and shRNA, and derivatives thereof.
